(19) United States
(12) Patent Application Publication
(10) Pub. No.: US 2020/0179337 A1
(43) Pub. Date: Jun. 11, 2020

(54) MEANS FOR TREATING SEXUALLY TRANSMITTED INFECTIONS

(71) Applicant: AKTSIONERNOE OBSCHESTVO "VELES FARMA", Moscow (RU)

(72) Inventor: Vsevolod Ivanovich KISELEV, Moscow (RU)

(73) Assignee: AKTSIONERNOE OBSCHESTVO "VELES FARMA", Moscow (RU)

(21) Appl. No.: 16/099,328

(22) PCT Filed: May 16, 2016
(86) PCT No.: PCT/RU2016/000293

§ 371 Date: Nov. 6, 2018

(51) Int. Cl. A61K 31/404; A61K 31/353; A61P 31/04

(52) U.S. Cl. CPC A61K 31/404; A61K 9/02; A61P 31/04; A61K 31/353

(57) ABSTRACT

Urogenital infections caused by microbial pathogens are treated by a pharmaceutical composition that treats or prevents a urogenital bacterial infection and that includes 3,3′-diindolylmethane (DIM), or a DIM-related indole, alone or in combination with epigallocatechin-3-gallate (ECGC) and additionally includes one or more antibacterial agents as an option. Such compositions and methods using same are especially useful for treatment or preventing bacterial infections, for example, Ureaplasma urealyticum, Mycoplasma spp, such as M hominis, N. gonorrhea, T. pallidum, Gardnerella spp., and Chlamydia spp., such as Chlamydia trachomatis. Treatment or prevention of any infection may be directed to the vagina, cervix, and/or uterus.

### FIELD OF THE INVENTION

The present invention relates to the treatment of urogenital infections caused by microbial pathogens. The present invention addresses such treatment by the combined administration of one, two or more pharmaceutically active agents.

### BACKGROUND TO THE INVENTION

Studies over the last ten years support the fact that, in 25-40% of cases, the cause of inflammatory diseases of the urogenital tract is genital Chlamydia, Mycoplasma or Ureaplasma spp. [1-3], the nature and outcome of the disease being largely determined by the state of reproductive system resistance factors. A study of the cellular and humoral factors of local immunity in infected women testifies to pronounced disorders of antimicrobial protection of the mucous membranes during urogenital infections, which in its turn explains the inadequate effectiveness of antibacterial therapy, prolonged and repeated courses of which lead to local immune dysfunctions [4-5]. The causative organisms of sexually-transmitted infections (STI) are defined as obligate pathogens. STIs have been found to play a significant role in the occurrence of female infertility and ectopic pregnancy [6-7]. There are no grounds for supposing that the problem of treating STIs will be resolved in the near future. The creation of novel highly-active antimicrobial drugs does not guarantee significant progress in enhancing the efficacy of treatment of female reproductive system diseases, which are primarily sexually transmitted. This fact is associated with the possibility of the causative organism persisting in a form which has low sensitivity to antibiotics.

3,3′-diindolylmethane (DIM) has previously been suggested for the treatment of inflammatory disease (see WO2006105196) and is shown to induce proapoptotic death of cells with disturbed metabolism, such as cells infected by intracellular organisms.

DIM and DIM-related indoles in combination with anti-inflammatory agents, such as epigallocatechin-3-gallate (EGCG) and/or antibacterial agents have been suggested for the treatment of oral mucosal disorders and promote bone health (see EP1865929).

DIM and EGCG have also been suggested for the treatment of cervical dysplasia, such as caused by human papilloma virus (HPV) infection (see WO2010027294).

It is amongst the objects of the present invention to provide compositions and methods of treating urogenital infections caused by microorganisms.

### SUMMARY OF THE INVENTION

In a first aspect, the present invention provides a pharmaceutical composition or compositions comprising DIM, or a DIM-related indole, alone or in combination with ECGC and optionally further comprising one or more anti-microbial agents, for use in a method of treating or preventing a urogenital infection caused by one or more microbes.

In a further aspect there is provided use of DIM, or a DIM-related indole, alone or in combination with ECGC and optionally further comprising one or more anti-microbial agents in the manufacture of a medicament for use in treating or preventing a urogenital infection caused by one or more microbes.

In a further aspect there is provided a method of treating or preventing a urogenital infection caused by one or more microbes, the method comprising administering a composition or compositions comprising DIM, or a DIM-related indole, alone or in combination with ECGC and optionally further comprising one or more anti-microbial agents, to a subject in need thereof.

According to the invention, the urogenital infection that is treated or prevented can result from, for example, a microbial (e.g., bacterial, viral, or fungal) infection. Methods and compositions of the present invention are particularly useful for treating or preventing infections caused by bacterial infections by, for example, Ureaplasma urealyticum, Mycoplasma spp, such as M hominis, N. gonorrhea, T. pallidum, Gardnerella spp., and Chlamydia spp., such as Chlamydia trachomatis, viral infections by, for example, Herpes Simplex, papilloma virus Cytomegalovirus, HIV, hepatitis, such as hepatitis B, or fungal infections by, for example, Candida albicans. Treatment or prevention of any infection may be directed to the vagina, cervix, and/or uterus, for example.

Suitable pharmaceutical compositions for urogenital application may be formulated as a suppository, an aqueous rinse, a cream, or a gel, and include at least DIM, or a DIM-related indole and/or ECGC and a pharmaceutically acceptable carrier. Desirably, DIM, or a DIM-related indole and ECGC are presented in the same formulation. Treatment using the compositions of the present invention may be self-administered. However, the compositions of the invention may be administered by a medical professional or other health care provider. Particularly useful pharmaceutical compositions may also contain a mucoadhesive or viscosity-enhancing agent.

The antimicrobial agent(s) can be present in the same or different pharmaceutical compositions as the other agents (i.e. DIM or DIM related compounds and ECGC), which themselves can be presented in a single or separate composition. When the antimicrobial agent(s) is/are present in a different pharmaceutical composition, different routes of administration may be used. For example, the antimicrobial agent(s) may be administered orally, or by intravenous, intramuscular, or subcutaneous injection. The antimicrobial agent(s) need not be administered intravaginally or intraurethrally. Frequently, for treating certain vaginal infections and sexually transmitted diseases, an antimicrobial agent(s) is/are administered orally and the other identified agents is/are administered intravaginally.

By “treating” is meant administering a pharmaceutical composition for prophylactic and/or therapeutic purposes.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

Preferred DIM-related indoles for use in the methods and compositions of the invention include, but are not limited to, hydroxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14- diindolylmethane.

The amount of said active agents to be administered should be such as to be a therapeutically effective amount. By “therapeutically effective amount” is meant an amount sufficient to provide medical benefit. When administering the compositions of the present invention to a human patient according to the methods described herein, a therapeutically effective amount is usually about 1-2500 mg of each active agent per dose. Preferably, the patient receives, 10 mg, 100 mg, 500 mg, 750 mg, 1000 mg, 1500 mg, or 2000 mg of each active agent in each dose. Dosing is typically performed 1-5 times each day, or less frequently, such as every 2, 3, 4, 5, 6 or 7 days.

Suppositories are solid dosage forms for insertion into the vagina for delivering medication to the vagina, cervix, and uterus. Typically, after insertion, the suppository softens, melts, disperses, or dissolves. Vaginal suppositories are usually about 0.5-7 grams each and may be tapered on both ends to facilitate application. Either a fatty or a water soluble/water miscible suppository base can be used in the compositions of this invention. Suitable fatty bases include, for example, cocoa butter, starch, such as potato starch, theobroma oil, vegetable oils modified by esterification, hydrogenation, glycerinated gelatin, and high molecular weight polyethylene glycols. Sustained release and/or prolonged contact of the therapeutics can be achieved by proper selection of a fatty suppository base material. Cocoa butter, for example, melts quickly at body temperature but is immiscible with body fluids, resulting in a prolonged but low level delivery of fat-soluble therapeutics to the affected sites. Alternatively, water soluble or water miscible bases (e.g., polyethylene glycols and glycol-surfactant mixtures) typically dissolve or disperse quickly, resulting in a rapid delivery of the therapeutic to the affected sites. An exemplary suppository formulation is described herein.

Other excipients may include one or more of the following: starch, for example corn starch, rice starch, potato starch, wheat starch, milk sugar (lactose), glucose, sucrose, micro-crystalline cellulose, colloidal silica, magnesium stearate, stearic acid, talc, polyvinylpyrrolidone (linear and cross-linked), sodium chloride, polyethylene glycol, hydroxypropyl-methylcellulose, hydroxypropylcellulose, gelatin, calcium phosphate, cellulose, mannitol, sodium carboxymethylstarch, sodium carbonate, sodium bicarbonate, calcium carbonate, sodium carboxymethylcellulose (linear and crosslinked) and magnesium stearate.

In an alternative formulation, one or more of the active agents can be encapsulated in biodegradable microspheres rather than being dissolved in the aqueous phase of the formulation. A wide variety of microencapsulation drug delivery systems have been developed and many share similar polymeric compositions as used for biodegradable films. Polymers commonly used in the formation of microspheres include, for example, methylacrylate polymers, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci., 68:1534, 1979).

Microspheres can be made by procedures well known in the art including spray drying, coacervation, and emulsification (see for example Davis et al. Microsphere and Drug Therapy, Elsevier, 1984; Benoit et al. Biodegradable Microspheres: Advances in Production Technologies, Chapter 3, Ed. Benita, S, Dekker, New York, 1996; Microencapsulation and Related Drug Processes, Ed. Deasy, Dekker, 1984, New York; U.S. Pat. No. 6,365,187). Preferably, the microspheres are bioadhesive or are prepared in formulations containing a bioadhesive excipient.

Other technical features of the compositions are easily modified to suit the specific pharmaceutical agent(s) and/or the clinical indication being treated. For example, the pH and/or osmolarity of the composition may be adjusted to confer stability, while minimizing vaginal and/or cervical irritancy and/or sensitivity.

Infection of the vaginal and cervical epithelium and of the external genitalia and the surrounding skin are amenable to compositions delivered as an ointment, paste, or gel. The viscous nature of these types of preparations allows for direct application. Such viscous formulations may also have a local barrier effect thereby reducing irritation and pain.

A mucoadhesive excipient can be added to any of the previously described pharmaceutical compositions. The mucoadhesive excipient may coat the relevant area, resulting in retention of said active agent(s) at the target site, providing protection, inhibiting irritation, and/or accelerating healing of inflamed or damaged tissue. Mucoadhesive formulations suitable for use in these pharmaceutical compositions are well known in the art (e.g., U.S. Pat. No. 5,458,879). Particularly useful mucoadhesives are hydrogels composed of about 0.05-20% of a water-soluble polymer such as, for example, poly(ethylene oxide), poly(ethylene glycol), poly(vinyl alcohol), poly(vinyl pyrrolidine), poly(acrylic acid), poly(hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel in the vagina, thereby providing a bioadhesive effect which acts to hold the therapeutic agents at the target site for an extended period of time. The anionic polysaccharides pectin and gellan are examples of materials which when formulated into a suitable composition will gel in the vagina, owing to the presence of cations in the mucosal fluids. The liquid compositions containing pectin or gellan will typically consist of 0.01-20% w/v of the pectin or gellan in water or an aqueous buffer system.

Other useful compositions which promote mucoadhesion and prolonged therapeutic retention in the vagina are colloidal dispersions containing 2-50% colloidal particles such as silica or titanium dioxide. Such formulations form as a flowable liquid with low viscosity suitable as a vaginal rinse; however, the particles interact with glycoprotein, especially mucin, transforming the liquid into a viscous gel, providing effective mucoadhesion (e.g., U.S. Pat. Nos. 5,993,846 and 6,319,513).

Furthermore, the compositions according to the invention may permit the treatment of so-called mixed infections caused by bacteria, viruses and/or fungi. When applying the compositions according to the invention, the plurality of pathogens are controlled simultaneously.

In optional embodiments of the methods and compositions, an antimicrobial agent(s) is/are included. Desirable antibacterial agents include, generally, penicillins, cephalosporins, tetracyclines, and aminoglycosides. Examples of antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

Antiviral agents are substances capable of destroying or suppressing the replication of viruses. Examples of anti-viral agents include 1,-D-ribofuranosyl-1,2,4-triazole-3 carboxamide, 9-(2-hydroxy-ethoxy) methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, amphotericin B, butylparaben, clindamycin, econaxole, fluconazole, flucytosine, griseofulvin, nystatin, clotrimazole, ketoconazole, enilconazole, itraconazole, butoconazole, and miconazole.

The antimicrobial agent may be administered within (either before or after administration of the other agents, 14 days, 7 days, 1 day, 12 hours, 4 hours, 2 hours, 1 hour, or substantially simultaneously with the other agents.

### DETAILED DESCRIPTION OF THE INVENTION

The present invention will now be further described by way of non-limiting example

### Clinical Characteristics of the Patient Groups

The study was carried out on 60 women with clinical signs of urogenital infections (cervicitis, urethritis, salpingo-oophoritis), which had been confirmed by the results of laboratory diagnosis. Vulvovaginitis of nonspecific aetiology also occurred in 12 cases, while a candido-urogenital co-infection was present in 14 women (detection of budding cells and/or pseudomycelium by microscopy and the growth of colonies of Candida spp. on inoculation of pathological material, and also isolation of the causative organisms of STI). The duration of the disease varied from 2 months to 4.5 years. The average age of the patients was 25.8±0.93 years and varied in the range from 19 to 50 years.

The patients were divided into 3 groups depending on the type of treatment of the urogenital infection. The groups were formed by random selection, ensuring uniformity in relation to age and the clinical signs of the disease.

Group 1 comprised 11 patients who received as the sole STI treatment vaginal suppositories with diindolylmethane and epigallocatechin as the active substances. The suppositories were prescribed one intravaginally every 24 h immediately after completion of menstruation, for a period of 20 days. 29 women (group 2) received combined treatment for the disease, including vaginal suppositories with diindolylmethane and epigallocatechin as the active substances, and standard antibacterial therapy. Depending on the causative organism of the inflammatory process identified: Chlamydia trachomatis, Ureaplasma parvum, Ureaplasma urealyticum, Mycoplasma hominis, Mycoplasma genitalium, Candida albicans, Papilloma virus infection, the patients were prescribed Wilprafen, 500 mg 3 times daily for 7 days; Macropen, 400 mg 3 times daily for 7 days; Zitrolid, 500 mg once daily for 8 days; Unidox solutab, 100 mg twice daily for 10 days; or Doxycycline, 0.1 g twice daily for 7 days. During the entire course of antibiotics, all patients received Nystatin, 500,000 IU 4 times daily for 10 days; Mycomax, a single dose of 150 mg; or Fluconazole, a single dose of 150 mg.

Group 3 comprised 20 patients, whose treatment did not include vaginal suppositories having diindolylmethane and epigallocatechin as the active substances. In addition to the above-mentioned antibacterial therapy, the patients of group 3 were, depending on the character of the mixed infection, prescribed Isoprinosine, 2 tablets 3 times daily for 14 days; Neo-Penetran suppositories, 1 suppository at night for 7 days; Hexicon suppositories, 1 suppository twice daily for 10 days; Genferon suppositories of 500 thousand IU twice daily for 10 days. The efficacy of the standard therapy in group 3 patients was assessed retrospectively.

### Investigational Methods

General clinical examination, microscopy of stained smears from the urethra, the cervical canal and the vagina, investigation of material using the polymerase chain reaction, cytological study of smears (the Pap-test), colposcopy, and pregnancy test.

### Characteristics of Vaginal Suppositories Having Diindolylmethane and Epigallocatechin as the Active Substances

The study material was in the form of vaginal suppositories containing 3,3′-diinidolylmethane and epigallocatechin 3-gallate:

| | |
|---|---|
| Epigallocatechin (FSP 42) | 0.150 g |
| Diindolylmethane | 0.150 g |
| Excipients | |
| Kolliphor 407 (EP 2007) | 0.280 g |
| Potato starch (GOST 7699-78, EP 2007, USP) | 0.014 g |
| Magnesium stearate | 0.004 g |
| (TU-6-09-16-1533-90, EP 2007) | |

Vaginal suppositories having diindolylmethane and epigallocatechin as the active substances are classed as virtually non-toxic preparations (LD50 more than 5 g/kg).

Pharmacokinetics: when used intravaginally, the active substances have high local bioavailability and virtually do not enter the blood circulation system.

Method of use and recommended doses: two regimes were employed for the preparation:

I—vaginal suppositories having diindolylmethane and epigallocatechin as the active substances were used intravaginally as the sole treatment in patients with urogenital infections, 1 suppository daily for 20 days.

II—as part of combined treatment of urogenital diseases comprising vaginal suppositories having diindolylmethane and epigallocatechin as the active substances and standard anti-microbial treatment.

### Statistical Analysis Methods

Variation statistics methods were used to process the results of the study. The parity of fractions was assessed using angular transformation (the Fisher φ-transformation) with the Yates continuity correction for comparable fractions.

### Results of the Study

The study was carried out on 60 women with clinical signs of urogenital infections (cervicitis, urethritis, salpingo-oophoritis), which had been confirmed by the results of laboratory diagnosis. The clinical characteristics of the patients are presented in Table 1.

TABLE 1
Clinical characteristics of patients with urogenital infections.

Assessment group (abs)

| Characteristic | 1 (n = 11) | 2 (n = 29) | 3 (n = 20) |
|---|---|---|---|
| Ureaplasma urealiticum | 1 | 8 | 2 |
| Chlamydia trachomatis | 4 | | 3 |
| Mycoplasma hominis | | | |
| Mycoplasma genitalium | | 1 | |
| HPV type 16 and 18 | 1 | 2 | |
| Gardnerella vaginalis | | | |
| Candidae fungi | | | |
| Ureaplasma urealiticum | | 3 | |
| HPV type 16 and 18 | | | |
| Mycoplasma hominis | | 1 | |
| HPV type 16 and 18 | | | |
| Ureaplasma urealiticum | | 4 | |
| Mycoplasma hominis | | | |
| Ureaplasma urealiticum | | | 3 |
| Candidae fungi | | | |
| Ureaplasma urealiticum | 1 | 1 | 2 |
| Gardnerella vaginalis | | | |
| Chlamydia trachomatis | | 1 | |
| HPV type 16 and 18 | | | |
| Chlamydia trachomatis | | | 1 |
| Candidae fungi | | | |
| Ureaplasma urealiticum | 1 | 1 | 1 |
| Chlamydia trachomatis | | | |
| Ureaplasma urealiticum | | 1 | |
| Mycoplasma hominis | | | |
| HPV type 16 and 18 | | | |
| Mycoplasma hominis | 1 | | 1 |
| Ureaplasma urealiticum | | | |
| Chlamydia trachomatis | | | |
| Ureaplasma urealiticum | | | 1 |
| HPV type 16 and 18 | | | |
| Candidae fungi | | | |
| Mycoplasma hominis | 1 | 1 | |
| Ureaplasma urealiticum | | | |
| Gardnerella vaginalis | | | |
| Chlamydia trachomatis | | | 1 |
| Ureaplasma urealiticum | | | |
| Gardnerella vaginalis | | | |
| Chlamydia trachomatis | | | 1 |
| HPV type 16 and 18 | | | |
| Candidae fungi | | | |
| Mycoplasma hominis | | 1 | |
| Ureaplasma urealiticum | | | |
| Chlamydia trachomatis | | | |
| HPV type 16 and 18 | | | |
| Ureaplasma urealiticum | | 1 | 1 |
| Chlamydia trachomatis | | | |
| HPV type 16 and 18 | | | |
| Candidae fungi | | | |
| Ureaplasma urealiticum | | 1 | 1 |
| Chlamydia trachomatis | | | |
| Mycoplasma hominis | | | |
| Candidae fungi | | | |
| Ureaplasma urealiticum | | 1 | 1 |
| Chlamydia trachomatis | | | |
| Mycoplasma hominis | | | |
| Candidae fungi | | | |
| HPV type 16 and 18 | | | |
| Ureaplasma urealiticum | 1 | 1 | 1 |
| Chlamydia trachomatis | | | |
| HPV type 16 and 18 | | | |
| Gardnerella vaginalis | | | |
| Candidae fungi | | | |

In 21 patients, the genital inflammatory process was caused by a monoinfection: in 11 women (18.3%) urogenital ureaplasmosis with a duration of more than 2 months was identified, while in 7 cases (11.7%) there was chronic urogenital chlamydiosis, 3 patients (5%) had type 16 and 18 HPV and 1 (1.7%) was infected with Mycoplasma hominis.

Mixed infections were diagnosed in 39 patients (65%). As concomitant genital infections, 14 patients (23.3%) were found to have genital candidosis and 7 (11.7%) bacterial vaginosis. In addition, 12 women (20%) had previously had signs of genital herpes. Infectious lesions of the genital organs, caused by three or more organisms, were observed in 18 (30%) of the subjects (Table 1).

Among the subjects, attempts had previously been made to treat the STI of 17 (28.3%) of the patients, including antimicrobial treatment and local interferon therapy. According to the history, the frequency of recurrences in the course of one year after treatment averaged 27.6%.

All patients were subjected to clinical examination, extended colposcopy, cytological study of smears, microscopy of Gram-stained smears, determination using the polymerase chain reaction of the presence of the following microorganisms: Chlamydia trachomatis, Ureaplasma parvum, Ureaplasma urealyticum, Mycoplasma hominis, Mycoplasma genitalium, Candida albicans and Papilloma virus infection, and investigation of material from the posterior vaginal vault using the culture method. According to the indications, a biopsy of the cervix was carried out followed by a morphological study of the biopsy material with van Gieson's stain. The content of human papilloma virus in material taken from foci of infection was determined in all patients, using the polymerase chain reaction with type-specific markers. At the same time, the type and quality of the oncogenic potential of the identified HPV were taken into account. Types 16, 18, 31, 33 and 35 were considered to be high oncogenic risk, and types 6 and 11 low oncogenic risk.

TABLE 2
Character and frequency of gynaecological diseases (abs, %)

| | Assessment group | | |
|---|---|---|---|
| Characteristic | 1 (n = 11) | 2 (n = 29) | 3 (n = 20) |
| Urethritis | 1 (9.1) | 8 (27.6) | 2 (10.0) |
| Cervicitis | 5 (45.5) | 1 (3.4) | 8 (40.0) |
| Vulvavaginitis | 1 (9.1) | 1 (3.4) | 1 (5.0) |
| Chronic inflammatory | 1 (9.1) | 1 (3.4) | 9 (45.0) |
| disease of internal | | | |
| genital organs | | | |
| Uterine myoma | | 1 (3.4) | 2 (10.0) |
| Genital endometriosis | 1 (9.1) | | 2 (10.0) |
| Primary and secondary sterility | 1 (9.1) | 1 (3.4) | 6 (30.0) |
| Menstrual function disorders | 2 (18.1) | 1 (3.4) | 1 (5.0) |
| Cervical ectopy (pseudo-erosion) | 5 (45.5) | 2 (6.9) | 8 (40.0) |
| Cervical dysplasia | 1 (9.1) | 2 (6.9) | 1 (5.0) |
| No gynaecological pathology | 4 (36.4) | 5 (17.2) | 5 (25.0) |

On initial examination, half of the subjects presented with various complaints: leukorrhoea worried 41% of the patients, vaginal itch 23%, and variously located pains 14%. In the majority of patients (78%), the duration of the cervical disease from the time of its identification was less than 5 years, while in patients with hypertrophic deformation of the cervix it was more than 10 years. An analysis of previous gynaecological pathology identified the presence of colpitis of varying aetiology in 21% of patients with cervical ectopy, and chronic inflammation of uterine appendages in 39%. Uterine myoma in the past was identified in 3 women, by reason of which one of the subjects had (2 years earlier) undergone supracervical hysterectomy. Four patients had in the past undergone hysteroscopy and separate diagnostic curettage for menstrual cycle disorder, and of these 2 were diagnosed with adenomyosis, and 1 with endometrial pathology, for which hormonal therapy for 3-6 months was recommended to all (with positive effect).

The clinical efficacy of the treatment carried out was assessed from the results of clinical and laboratory examination. Microscopic examination of smears from the urethra, the cervical canal and the vagina, with Romanowsky-Giemsa and Gram staining, investigation of material from the cervical canal and urethra using the polymerase chain reaction, and investigation of material from the posterior vaginal vault by culturing, were carried out before starting the treatment and 3 weeks after its completion.

TABLE 3
Extent of urogenital infection before and after treatment

| | Assessment groups | | | | | |
|---|---|---|---|---|---|---|
| | Group I (n = 11) | | Group II (n = 20) | | Group III (n = 29) | |
| Organism causing | Before | After | Before | After | Before | After |
| infection | treatment | treatment | treatment | treatment | treatment | treatment |
| Ureaplasma urealyticum | 6 | 5 | 20 | 0 | 15 | 7 |
| Chlamydia trachomatis | 7 | 6 | 6 | 0 | 13 | 0 |
| Mycoplasma hominis | 2 | 0 | 7 | 0 | 4 | 3 |
| Candida spp. | 2 | 0 | 6 | 0 | 6 | 13 |
| Gardnerella vaginalis | 3 | 0 | 2 | 0 | 4 | 4 |
| HPV type 16 and 18 | 2 | 1 | 9 | 3 | 5 | 4 |

The confidence level of the results of the investigations was confirmed by medical statistics methods using the Student t-test and the X²-test.

### CONCLUSION

Analysis of the results of the study has made it clear vaginal suppositories having diindolylmethane and epigallocatechin as the active substances had a substantial positive influence on the recovery process, both for patients with a single agent infection caused by Chlamydia trachomatis, Ureaplasma urealyticum, Mycoplasma hominis, Mycoplasma genitalium, Gardnerella vaginalis or HPV, and also where mixed infections are present. The effect of the preparation was a statistically significant (3.5-fold) fall in the frequency of recurrences of the disease and the need to prescribe repeat courses of treatment. In patients with frequently recurring inflammatory diseases of the genitalia, who had repeatedly received standard antimicrobial treatment, the efficacy of the combined treatment rose to 96.7%, while with conventional (antimicrobial?) treatment the efficacy of treatment is 55.0% (p<0.001).

The antimicrobial treatment used to treat gynaecological diseases may at the same time lead to disruption of the micro-ecology of other biotopes of the human body or may enhance the extent of already existing disruptions of the micro-ecology of the reproductive system. A conventional treatment regime with the use of metronidazole and clindamycin was employed in the control group of patients with the object of correcting dysbiotic disorders of the vaginal microflora, and against this background a fall was observed in the number of aerobic and anaerobic conditionally pathogenic microorganisms. However, over the course of a month a lactoflora deficit was maintained and an increase of 17% occurred in the number of yeast-like Candida fungi, while in 23% complications occurred after treatment (nausea, vomiting, stomach pain, etc.).

The results of the present study have demonstrated a fall in the number of aerobic and anaerobic conditionally pathogenic microorganisms when using vaginal suppositories having diindolylmethane and epigallocatechin as the active substances both as a monotherapy and also as part of combined treatment. In the course of local treatment with the vaginal suppository of the present invention virtually no complications were recorded, the microbiocenosis conformed to the norm, and normalization of the number of lactobacilli (104-5 or more) was observed. No yeast-like Candida fungi were isolated a month after treatment. The use of the vaginal suppository therapy in gynaecological practice thus has the advantage over conventional treatment methods. In this case, the elimination not only of pathogenic but also of conditionally-pathogenic microflora proceeds more effectively, with an increase in the number of lactobacilli.

At 27.3% (p<0.001), the efficacy of treatment was lowest in the group of patients who were given the vaginal suppository as the sole treatment for STI. However, in all patients with ectopy included in the main assessment groups, the results of colposcopy and cytological examination of the cervix showed a positive effect in the condition of the cervix. 3-4 weeks after treatment, a full effect was identified in 45.5% of patients with ectopy who had received the vaginal suppository as the sole treatment, and in 80.0% (p<0.01) of women on a background of combined treatment. Individual analysis showed that the treatment was ineffective in patients with a long history of the disease (more than 5 years from the time of diagnosis) and also in patients previously treated by conservative methods with partial effect. Treatment with the vaginal suppository was ineffective in 28% of patients with superficial nabothian cervical cysts. At the same time, in all patients with a prior diagnosis of cervical dysplasia and who were treated using the vaginal suppository, this diagnosis was not confirmed on cytological investigation of the cervix after treatment. Without wishing to be bound by theory the efficacy may be associated with the anti-oestrogenic action of the vaginal suppository on the cervix, which stimulates the activity of the cytochrome CYP450 1A1 isoenzyme, shifting the equilibrium towards 2-hydroxy-oestrone, which facilitates the death of tumour cells and prevention of their further formation; it competes with oestrogens for bonding with receptors, and reduces the number of oestrogen receptors on the target cells.

Thus, on the basis of clinical and laboratory monitoring, the results of the study confirm the effectiveness of including vaginal suppository in the combined treatment employed and allow the preparation to be recommended in combined treatment of urogenital infections with chronic chlamydiosis, ureaplasmosis, mycoplasmosis, gardnerellosis and PVI, and also in combinations of these. Taking account of the high oncogenic potential of HPV in women, the vaginal suppository must be regarded as a means for prophylaxis of proliferative processes in the cervix. With the object of rational pharmacotherapy, it is desirable to use the vaginal suppository in the combined treatment of background and precancerous diseases of the cervix as being a preparation which is highly efficacious and does not have side-effects.

### Results of the Study of Vaginal Suppositories Comprising 3,3′-diindolylmethane (DIM) as an Active Agent

The studied material was vaginal suppositories comprising 3,3′-diindolylmethane (DIM):

| | | |
|---|---|---|
| Diindolylmethane | 0.2 | g |
| Excipients | | |
| Kolliphor 407 (EP 2007) | 0.280 | g |
| Potato starch (GOST 7699-78, EP 2007, USP) | 0.014 | g |
| Magnesium stearate | 0.004 | g |

Two modes of administration recommended doses of the medication were prepared:

I—vaginal suppositories, comprising 3,3′-diindolylmethane (DIM) as an active agent, were to be administered vaginally as an alone therapeutic agent 1 (one) suppository a day during 20 days for patients with urogenital infections.

II—as a part of a combined treatment of urogenital infection, including administering of vaginal suppositories, comprising 3,3′-diindolylmethane (DIM) as an active agent and standard antimicrobial treatment.

30 women with clinical signs of inflammatory process in the urogenital system caused by mixed infection took part in the study. Infectious lesions of genital organs caused by three or more microorganisms were noted in 22 (73%) women. All patients were divided into two groups formed at random providing uniformity in respect to age and clinical signs of diseases.

Group 1 included 10 patients who received vaginal suppositories, comprising 3,3′-diindolylmethane (DIM) as an active agent, as an alone therapeutic agent. The suppositories were administered vaginally by 1 (one) every 24 hours, immediately after the end of menstruation during 20-days period.

Group 2 included 10 patients. They received a combined treatment, including administering of vaginal suppositories, comprising 3,3′-diindolylmethane (DIM) as an active agent and standard antibacterial treatment. Depending upon the identified microorganism causing an inflammatory process: Chlamydia trachomatis, Ureaplasma parvum, Ureaplasma urealyticum, Mycoplasma hominis, Mycoplasma genitalium, Candida albicans, Papilloma virus infection, the patients were prescribed Wilprafen, 500 mg, 3 times daily for 7 days; Macropen, 400 mg, 3 times daily for 7 days; Zitrolid, 500 mg, once daily for 8 days; Unidoxsolutab, 100 mg, twice daily for 10 days or Doxycyclinum, 0.1 g twice daily for 7 days. Throughout the course of antibiotic treatment all patients received Nystatinum, 500,000 ME 4 times daily for 10 days; Mycomax, a single dose 150 mg or Fluconazolum, a single dose 150 mg.

Group 3 included 10 patients for whom vaginal suppositories, comprising 3,3′-diindolylmethane (DIM) as an active agent, were not used. All patients received above mentioned antibacterial therapy.

TABLE 4
Results of evaluation of effectiveness of therapy

| Effect of | Group 1 | Group 2 | Group 3 |
|---|---|---|---|
| therapy | (n = 10) | (n = 10) | (n = 10) |
| No | 1 | 0 | 3 |
| Yes | 9 | 10 | 7 |

1-15. (canceled)

16. A pharmaceutical composition for treating or preventing a urogenital bacterial infection comprising 3,3′-diindolylmethane (DIM), or a DIM-related indole, alone or in combination with epigallocatechin-3-gallate (ECGC).

17. The pharmaceutical composition according to claim 16 comprising additionally one or more antibacterial agents.

18. The pharmaceutical composition according to claim 16 for treating or preventing infections caused by bacteria, for example, Ureaplasma urealyticum, Mycoplasma spp, such as M hominis, N. gonorrhea, T. pallidum, Gardnerella spp., and Chlamydia spp., such as Chlamydia trachomatis.

19. The pharmaceutical composition according to claim 16, wherein DIM, or DIM-related indole alone or in combination with ECGC are formulated as a suppository, an aqueous rinse, a cream, or a gel.

20. The pharmaceutical composition according to claim 16, wherein the DIM-related indole includes, but is not limited to, hydroxylated DIM, methoxylated DIM, 2-(Indol-3-ylmethyl)-3,3′-diindolylmethane (LTR), hydroxylated LTR, methoxylated LTR, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14- diindolylmethane.

21. The pharmaceutical composition according to claim 16, wherein the antibacterial agent(s) includes, generally, penicillin, cephalosporin, tetracycline, aminoglycoside, macrolide, fluoroquinolone or other antibiotic.

22. The pharmaceutical composition according to claim 21, wherein the penicillin is penicillin G, ampicillin, methicillin, oxacillin, or amoxicillin.

23. The pharmaceutical composition according to claim 21, wherein the cephalosporin is cefadroxil, ceforanid, cefotaxime, or ceftriaxone.

24. The pharmaceutical composition according to claim 21, wherein the tetracycline is doxycycline, minocycline, tetracycline, or oxytertracycline.

25. The pharmaceutical composition according to claim 21, wherein the aminoglycoside is amikacin, gentamycin, kanamycin, neomycin, streptomycin, or tobramycin.

26. The pharmaceutical composition according to claim 21, wherein the macrolide is azithromycin, clarithromycin, leucomycin and erythromycin.

27. The pharmaceutical composition according to claim 21, wherein the fluoroquinolone is ciprofloxacin, lomefloxacin, and norfloxacin.

28. The pharmaceutical composition according to claim 21, wherein the other antibiotic includes chloramphenicol, clindamycin, cycloserine, isoniazid, rifampin, azithromycin or vancomycin.

29. A use of the pharmaceutical composition according to claim 16 in the manufacture of a medicament for use in treating or preventing a urogenital bacterial infection caused by one or more microbes.

30. A method of treating or preventing a urogenital infection caused by one or more microbes, the method comprising administering the pharmaceutical composition according to claim 16 to a subject in need thereof.

31. The method according to claim 30, comprising administering of a pharmaceutical composition comprising 3,3′-diindolylmethane (DIM), or a DIM-related indole, alone or in combination with epigallocatechin-3-gallate (ECGC) and additionally one or more antibacterial agents.

32. The method according to claim 30, wherein additionally the antimicrobial agent(s) is administered orally, or by intravenous, intramuscular, or subcutaneous injection.
